(19) United States
(12) Patent Application Publication
(10) Pub. No.: US 2009/0306497 A1
(43) Pub. Date: Dec. 10, 2009

(54) CALIBRATION METHOD FOR CATHETER TRACKING SYSTEM USING MEDICAL IMAGING DATA

(75) Inventors: Robert Manzke, Cambridge, MA (US); Raymond Chan, Brookline, MA (US); Shunmugavelu Sokka, Brighton, NY (US); Volker Rasche, Erbach (DE)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/301,312

(22) PCT Filed: Apr. 10, 2007
(86) PCT No.: PCT/IB2007/051285

§ 371 Date: Jul. 17, 2009

Related U.S. Application Data
(60) Provisional application No. 60/803,247, filed on May 26, 2006.

(51) Int. Cl. A61B 5/06; A61B 6/12

(52) U.S. Cl. 600/424

(57) ABSTRACT

This invention discloses an improved method for the calibration and tracking of an electromagnetic or acoustic based catheter within a catheter tracking space for use in cardiac intervention for a specific patient, utilizing prior-acquired medical imaging data for the patient.

The invention relates to a method for improving the calibration and tracking of catheters in cardiac intervention using prior-acquired medical image data.

Medical diagnostic and imaging systems are present in modern health care facilities. Such systems provide invaluable tools for identifying, diagnosing and treating physical conditions and greatly reduce the need for surgical diagnostic intervention. In many instances, final diagnosis and treatment proceed only after an attending physician or radiologist has complemented conventional examinations with detailed images of relevant areas and tissues via one or more imaging modalities.

With the increasing use of minimally invasive surgical techniques in medical diagnosis and therapy, there is a need for a new method of remotely locating and tracking catheters or other medical instruments inside a human or animal body. Currently, x-ray fluoroscopic imaging is the standard catheter tracking technique. However, excessive exposure to x-ray dosages by both the patient and clinician can be harmful. Thus, alternative catheter tracking methods are desirable.

Several alternative methods have been published including some which employ ultrasonic transducers and others which make use of magnetic field measurements.

One known method of catheter location employs one or more magnetic field sources, which are fixed relative to one another and define a spatial reference frame, and one or more magnetic sensors, fixed to the tip of the catheter. The sensors measure the fields produced by the sources, and these measurements are then used to determine the tip's position relative to the reference frame. The same result could alternatively be achieved with the sources replaced by sensors, and the sensors by sources.

This technique relies on accurate prior knowledge of the relative positions of the sources and the spatial forms of their magnetic fields, and of the relative positions and sensitivities of the sensors. Because it is not possible to manufacture sources and sensors with ideal characteristics, purely theoretical calculations of such characteristics are likely to be erroneous, and hence they must be determined from calibration measurements. One advantage of using magnetic fields to track a catheter inside a human or animal body is that the fields are virtually unaffected by the presence of the body. This is due to the very low magnetic susceptibility of body tissue. In contrast, electric and acoustic fields are strongly affected by body tissue. The result is that calibration measurements of a magnetic field tracking system can be made without the presence of the body, before surgery.

A limitation placed on catheters is that they must be small enough in diameter and flexible enough to allow insertion into the relevant part of the body. For example, cardiac catheter diameters should be around 2 mm, and flexible enough to bend to a radius of 10 mm or less. These requirements, and the need to fix the catheter mounted transducers rigidly together, close to the catheter head, demand that these transducers must all be contained in a small volume.

Currently, electrical-field-based catheter tracking systems such as the NavX (ESI, St. Jude) and the Localisa system (Medtronic) or ultrasound-time-of-flight-based tracking systems rely on the assumption of homogeneous and linear electromagnetic fields or uniform speed-of-sound distributions within the mediastinum during the calibration and data acquisition processes. However, the spatial distribution of electrical conductivity and acoustic properties inside the human thorax changes depending on the anatomy of the patient and dynamic effects such as respiration. Hence, the intrathoracic electromagnetic and acoustic fields are by no means homogeneous and linear, leading to large errors during the catheter tracking. At the moment, such tracking devices are used only within anatomical structures in which the electromagnetic or speed-of-sound field is approximately homogeneous such as within the blood pool in the cardiac chambers. Simple linear calibration methods are used in order to perform tracking of the catheter depending on measuring either the electrical field or ultrasound-time-of-flight in the body by means of electromagnetic or ultrasonic sensors on the catheter. These techniques have not yet been applied successfully within other anatomical structures, such as the cardiac veins, where field in homogeneity and non-linearity preclude reliable tracking with current linear field calibration techniques.

Catheter tracking and calibration systems have been described in U.S. Pat. No. 5,697,377 issued Dec. 16, 1997; U.S. Patent Application Numbers 20050080328 published on Apr. 14, 2005; 20060058643 published Mar. 16, 2006; and 20050197568 published Sep. 8, 2005; U.S. Pat. No. 6,226,547 issued May 1, 2001; PCT International publication Number WO 2005/048841 published Jun. 2, 2005; and in the published article “A System for Real-Time XMR Guided Cardiovascular Intervention”, K. S. Rhode et al., IEEE Trans. Medical Imaging, 24, (11), November 2005, pp 1428-1440.

However, problems still persist with these techniques. Currently, electrical-field-based catheter tracking systems such as the NavX (ESI, St. Jude) and the Localisa system (Medtronic) rely on the assumption of homogeneous and linear electric fields during the calibration and data acquisition processes. However, the electrical conductivity inside the human body, especially in the chest changes depending on patient-specific anatomy and other dynamic effects such as respiratory motion. The electrical field is therefore non-homogeneous and non-linear, leading to significant errors during the catheter tracking. Likewise, ultrasound-based position sensing assumes homogeneity of the speed of sound within tissue. The non-uniform distribution of the speed-of-sound within the chest distorts ultrasound-derived position and orientation measurements leading to positioning errors within the volume.

According to this invention, a novel approach is presented which uses patient-specific imaging data from either MDCT or MR for segmentation of the major mediastinal structures and construction of multi-dimensional conductivity or acoustic models, which can then be used for calculations of the electrical or acoustic field in order to improve the calibration process and the tracking of catheters.

It is an object of this invention to provide a method for improving the calibration and tracking of an electromagnetic or acoustic based catheter within a catheter tracking space for use in cardiac intervention for a specific patient comprising: acquiring before or during the intervention a volume of cardiac image data for a measuring space of the patient from a medical scanner; segmenting the image data according to tissue region to generate medical images of each tissue region; acquiring the electromagnetic or acoustic data of each tissue region; processing the electromagnetic or acoustic data to generate an electromagnetic or acoustic model image for one or more segmented tissue regions covering a model space corresponding to the measuring space of the patient and the catheter tracking space; wherein the model image indicates one or more areas of signal distortion within the catheter tracking space affecting the accuracy of the data; registering the model space with the catheter tracking space; measuring the tracking behavior of the catheter during intervention and with reference to the model image to determine catheter tracking errors resulting from signal distortion effects; and correcting the position of the catheter during tracking to minimize the effects of signal distortion.

Another object is to provide a method wherein the acquiring before the intervention a volume of cardiac image data further comprises: acquiring before the intervention a volume of cardiac image data using at least one of a CT system, a MR system, an Ultrasound system, a 3D Fluoroscopy system, and a PET system.

Another object is to provide a method wherein the tissue region further comprises: at least one of the cardiac veins, cardiac arteries, or aorta.

Another object is to provide a method wherein the processing the electromagnetic data to generate an electromagnetic model image further comprises: processing the electromagnetic data to generate a 4D electrical conductivity model image.

Another object is to provide a method wherein the processing the acoustic data to generate an acoustic model image further comprises: processing the acoustic data to generate a 3D speed-of-sound model image.

Another object is to provide a method wherein the registering the model space with the catheter tracking space further comprises: registering the model space with the catheter tracking space using electromagnetic or acoustic visible surface markers attached to the patient within the model space, wherein the position of each surface marker corresponds to the placement of reference patches placed at the marked positions in the catheter tracking space during catheter calibration or tracking.

These and other aspects of the invention are explained in more detail with reference to the following embodiments and with reference to the FIGURES.

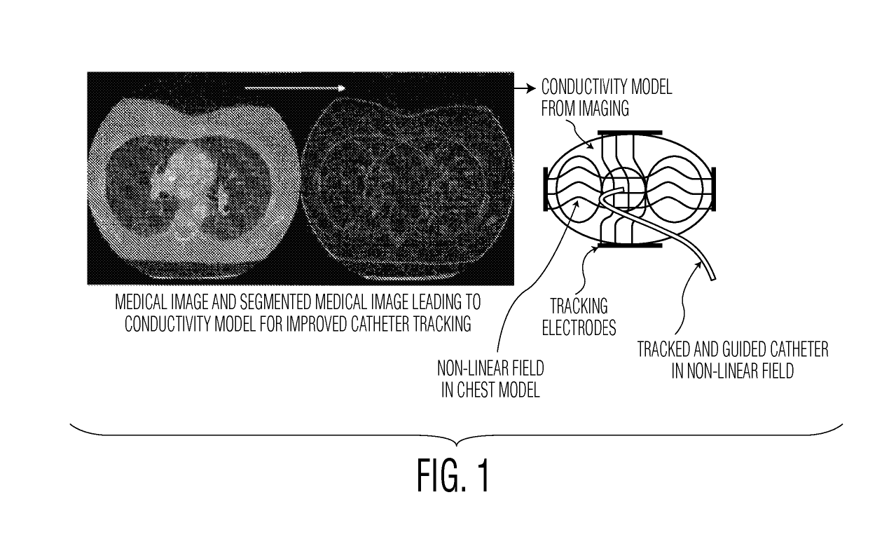

FIG. 1 shows a medical image and segmented medical image leading to conductivity model for improved catheter tracking.

Cardiovascular catherization procedures are traditionally carried out under X-ray fluoroscopic guidance. This type of guidance has several disadvantages. First, since the images produced are two dimensional (i.e., 2D), there is a need to take several picture from different angles to try to reconstruct a three dimensional (i.e., 3D) image of the object being imaged with inherent inaccuracies occurring in visualizing the 3D path of the catheter. Second, X-rays do not accurately pick up the images of soft tissue regions such as the heart and blood vessels. Third, X-ray imaging exposes the operator and patient to radiation doses, which over time can cause significant health problems.

Currently, electrical-field-based catheter tracking systems such as the NavX (ESI, St. Jude) and the Localisa system (Medtronic) rely on the assumption of homogeneous and linear electric fields during the calibration and data acquisition processes. However, the electrical conductivity inside the human body, especially in the chest changes depending on patient-specific anatomy and other dynamic effects such as respiratory motion. The electrical field is therefore non-homogeneous and non-linear, leading to significant errors during the catheter tracking. Likewise, ultrasound-based position sensing assumes homogeneity of the speed of sound within tissue. The non-uniform distribution of the speed-of-sound within the chest distorts ultrasound-derived position and orientation measurements leading to positioning errors within the volume.

According to the prior art, systems such as the St. Jude NavX rely on tracking a catheter in an electromagnetic field. Therefore, 3 orthogonal fields are applied to the patient by means of body surface electrodes as is described in, for example, U.S. Pat. No. 5,697,377 to Wittkampf. Since the impedance within the body varies (lungs, different tissue types etc.) the e-field in the body is strongly non-linear. A tracked catheter can be maneuvered well to similar positions but absolute measurement cannot be performed. Hence, mathematical models of the anatomy generated from point clouds of the tracked catheter are distorted. Also, the tracking can only be performed in certain anatomic regions such as cardiac chambers. Instead, in blood vessels or arteries the tracking results are compromised by large errors due to the non-homogeneity of the medium and large field distortions.

In this invention, a novel approach is presented which uses patient-specific imaging data from either MDCT or MR for segmentation of the major mediastinal structures and construction of multi-dimensional conductivity or acoustic models, which can then be used for calculations of the electrical or acoustic field in order to improve the calibration process and the tracking of catheters.

In this invention, imaging data can be used to deconvolute this non-linear space and make tracking more accurate and feasible in other anatomic regions such as blood vessels. In a clinical workflow, imaging data could be obtained, for example, at the beginning of the intervention, such data assigned with typical body impedances and subsequently used for unwarping the space of the catheter tracking. If intra-interventional imaging is available, the unwarping can be updated according to such data. Pre-operation data can be obtained by modalities such as CT/MRI/X-ray/US with the corresponding settings for cardiac data acquisition.

This invention disclosure proposes to use pre-acquired, multi-dimensional, patient-specific data obtained using imaging with MRI or CT to construct electrical conductivity or speed-of-sound models for improving the calibration of catheter tracking systems. Electrical conductivity models have already been used in the field of electric impedance tomography. Reliable tracking in areas with rapidly changing electromagnetic or acoustic field properties such as the cardiac veins and arteries, the aorta etc. can become feasible using improved calibration and tracking strategies based on the use of such patient-specific models. Dynamic effects such as respiration (volume changes of the lung leading to anatomical changes and associated changes in conductivity and speed-of-sound distributions) can be accounted for using a four-dimensional chest model derived from medical imaging data.

A multi-dimensional conductivity or speed-of-sound model of, for example, the chest is derived from medical imaging data, as is shown in FIG. 1. This data is used to predict the electromagnetic or acoustic field within the anatomy of interest. The calibration of electromagnetic or acoustic field based catheter tracking systems is improved using these patient-specific models and tracking can be performed more reliably in areas with field distortions due to rapid changing anatomical structure and tissue properties. Patient motion can be accounted for, leading to a reduction of tracking errors.

While the present invention has been described with respect to specific embodiments thereof, it will be recognized by those of ordinary skill in the art that many modifications, enhancements, and/or changes can be achieved without departing from the spirit or scope of the invention. Therefore, it is manifestly intended that the invention be limited only by the scope of the claims and equivalents thereof.

1. A method for improving the calibration and tracking of an electromagnetic or acoustic based catheter within a catheter tracking space for use in cardiac intervention for a specific patient comprising:
acquiring before or during the intervention a volume of cardiac image data for a measuring space of the patient from a medical scanner;
segmenting the image data according to tissue region to generate medical images of each tissue region;
acquiring the electromagnetic or acoustic data of each tissue region;
processing the electromagnetic or acoustic data to generate an electromagnetic or acoustic model image for one or more segmented tissue regions covering a model space corresponding to the measuring space of the patient and the catheter tracking space;
wherein the model image indicates one or more areas of signal distortion within the catheter tracking space affecting the accuracy of the data;
registering the model space with the catheter tracking space;
measuring the tracking behavior of the catheter during intervention and with reference to the model image to determine catheter tracking errors resulting from signal distortion effects; and
correcting the position of the catheter during tracking to minimize the effects of signal distortion.

2. The method of claim 1 wherein the acquiring before the intervention a volume of cardiac image data further comprises:
acquiring before the intervention a volume of cardiac image data using at least one of a CT system, a MR system, an Ultrasound system, a 3D Fluoroscopy system, and a PET system.

3. The method of claim 1 wherein the tissue region further comprises:
at least one of the cardiac veins, cardiac arteries, or aorta.

4. The method of claim 1 wherein the processing the electromagnetic data to generate an electromagnetic model image further comprises:
processing the electromagnetic data to generate a 4D electrical conductivity model image.

5. The method of claim 1 wherein the processing the acoustic data to generate an acoustic model image further comprises:
processing the acoustic data to generate a 3D speed-of-sound model image.

6. The method of claim 1 wherein the registering the model space with the catheter tracking space further comprises:
registering the model space with the catheter tracking space using electromagnetic or acoustic visible surface markers attached to the patient within the model space, wherein the position of each surface marker corresponds to the placement of reference patches placed at the marked positions in the catheter tracking space during catheter calibration or tracking.
